(19) United States
(12) Patent Application Publication
(10) Pub. No.: US 2025/0302483 A1
(43) Pub. Date: Oct. 2, 2025

(54) SHUNT BLEED INHIBITOR BANDAGE

(71) Applicant: Cole Kevin Jahn Stebbins, Silverado, CA (US)

(72) Inventor: Cole Kevin Jahn Stebbins, Silverado, CA (US)

(21) Appl. No.: 18/619,621

(22) Filed: Mar. 28, 2024

(51) Int. Cl. A61B 17/132; A61B 17/12

(52) U.S. Cl. CPC A61B 17/132; A61B 2017/12004

(57) ABSTRACT

According to the present invention disclosed herein, there is provided device for managing shunt bleeds, comprising: a strap; a dome part; and an adhesive, wherein the adhesive is applied to attach the dome part on top of a shunt and tightly seal the dome part to the patient's skin so that blood cannot escape from the dome.

### BACKGROUND

Devices and methods to stop bleeding have been disclosed in the prior art patent literature.

For example, U.S. Pat. No. 10,058,334 discloses a method to stop bleeding at a puncture site of an artery on a patient's arm including wrapping a flexible band of a hemostatic device around at least a portion of the patient's arm, wherein the hemostatic device also comprises a more rigid member that is more rigid than the flexible band, a balloon, and a visually identifiable marker. The method also involves wrapping the flexible band around at least the portion of the patient's arm while the balloon is in an uninflated state and while an introducer sheath is in place at the puncture site. The visually identifiable marker is used to position the balloon in overlying relation to the puncture site while viewing the puncture site through the balloon and through the more rigid member. Fluid is introduced into the balloon to inflate the balloon to apply a compression force against the puncture site.

U.S. Pat. No. 6,791,723 discloses a tamponade catheter for controlling bleeding from a penetrating or perforating wound in a patient comprising a catheter having an inner catheter lumen with a non-elastic inflatable balloon secured to the catheter in fluid communication with an inner catheter lumen of the catheter. A removable outer sheath overlays the inflatable balloon for inserting the inflatable balloon within the wound of the patient. A bendable flexible stiffening stylet is receivable within the inner catheter lumen of the catheter to aid in navigating the catheter into the wound of the patient. The removable outer sheath and the stylet are removable for inflating the inflatable balloon by an introduction of a fluid through the inner catheter lumen of the catheter for enabling the inflated non-elastic inflatable balloon to create pressure within the wound of the patient to control bleeding therefrom.

However, prior art devices and methods do not work well for shunt bleeds, which are a significant problem for many patients, i.e., dialysis patients.

### SUMMARY OF INVENTION

Therefore, the present invention provides a device which is optimized for control of shunt bleed.

According to an aspect of the present invention disclosed herein, there is provided a device for managing shunt bleeds, comprising: a strap; a dome part; and an adhesive, wherein the adhesive is applied to attach the dome part on top of a shunt and tightly seal the dome part to the patient's skin so that blood cannot escape from the dome.

### BRIEF DESCRIPTION OF THE DRAWINGS

FIG. 1 is an illustration of a top view of a device according to an embodiment of the present invention.

FIG. 2 is an illustration of a cross-sectional view of the device.

### DETAILED DESCRIPTION

The present invention is a shunt bleed management device which has a dome for collecting blood and an adhesive which securely attaches the dome around the shunt so that blood pools under the dome and pressure is applied on the opening of the shunt to prevent further bleeding.

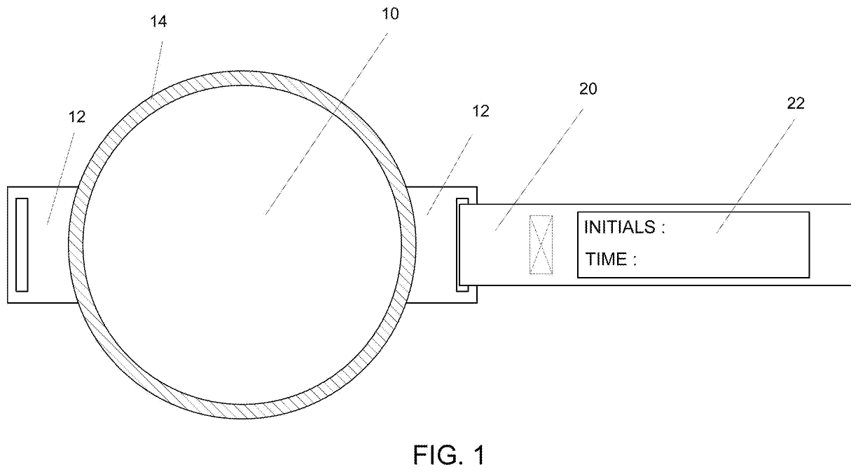

FIG. 1 is an illustration of a top view of a device according to an embodiment of the present invention.

The device consists of the following components:
10—refers to the device as a whole
12—dome part for containing blood
14—adhesive
20—strap for attachment
22—writing area for information on placement time.

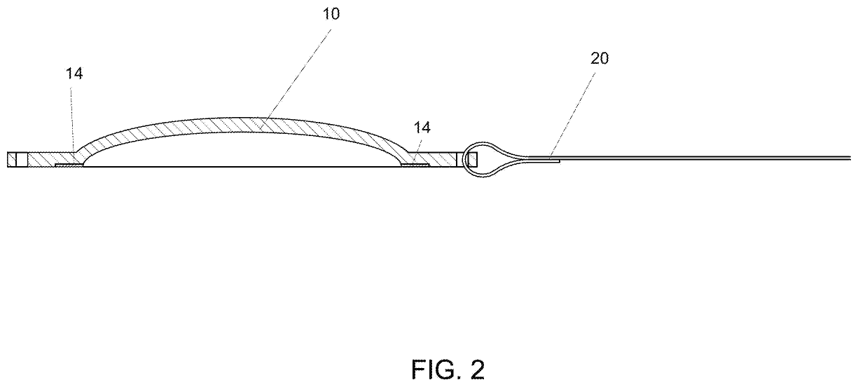

FIG. 2 is an illustration of a cross-sectional view of the device.

The cross-sectional view illustrates the following components:
10—refers to the device as a whole
12—dome part for containing blood
14—adhesive
20—strap for attachment
22—writing area for information on placement time.

The mechanism stopping the bleeding is creating an equilibrium of pressure inside the device and the puncture site with a complete seal thus negating blood loss.

The band used in the present invention is not stretchable, and this is a key advantage over the prior art.

There is nowhere else for the fluid to go to so the pressure in the dome becomes the same as the shunt, stopping the further pressure increase of the dome and stopping further blood loss. The pressure will not continue to build, it can only max at what the pressure of the body is.

Without the dome it would continue to bleed out of the arm as it is not going to clot. It's essentially like putting a water bottle cap over a leaking hole. The hole now won't leak, unlike putting a trauma dressing over the hole, where it would just soak through and continue to bleed.

The illustrations of embodiments described herein are intended to provide a general understanding of the structure of various embodiments, and they are not intended to serve as a complete description of all the elements and features of apparatus and systems that might make use of the structures described herein.

Many other embodiments will be apparent to those of skill in the art upon reviewing the above description. Other embodiments may be utilized and derived therefrom, such that structural and logical substitutions and changes may be made without departing from the scope of this disclosure.

Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed.

What is claimed is:

1. A device for managing shunt bleeds, comprising:
a strap;
a dome part; and
an adhesive, wherein the adhesive is applied to attach the dome part on top of a shunt and tightly seal the dome part to the patient's skin so that blood cannot escape from the dome.

2. The device for managing shunt bleeds of claim 1, further comprising a label.

3. The device for managing shunt bleeds of claim 1, wherein the label contains a field to indicate the date and time the device was placed.

4. The device for managing shunt bleeds of claim 1, wherein the adhesive is Tegaderm.

5. The device for managing shunt bleeds of claim 1, wherein the dome part is plastic.
